(19) United States
(12) Patent Application Publication
(10) Pub. No.: US 2022/0061704 A1
(43) Pub. Date: Mar. 3, 2022

(54) APPARATUS AND METHOD FOR ESTIMATING TARGET COMPONENT

(71) Applicant: SAMSUNG ELECTRONICS CO., LTD., Suwon-si (KR)

(72) Inventors: June Young LEE, Seongnam-si (KR); Yun S Park, Suwon-si (KR)

(73) Assignee: SAMSUNG ELECTRONICS CO., LTD., Suwon-si (KR)

(21) Appl. No.: 17/117,987

(22) Filed: Dec. 10, 2020

(30) Foreign Application Priority Data
Sep. 1, 2020 (KR) 10-2020-0110929

(51) Int. Cl. A61B 5/1455; A61B 5/145; A61B 5/00; G06N 20/00; G16H 40/63

(52) U.S. Cl. CPC A61B 5/1455; A61B 5/14532; G16H 40/63; A61B 5/7267; G06N 20/00; A61B 5/14546

(57) ABSTRACT

Provided is an apparatus configured to estimate a target component, the apparatus including a spectrum acquisition device configured to acquire a training spectrum, and a processor configured to extract a principal component from the acquired training spectrum, determine an optimal number of principal components based on a size of the extracted principal component and a size of a residual, and generate a target component estimation model based on the determined optimal number of principal components.

### CROSS-REFERENCE TO RELATED APPLICATION

This application claims priority to Korean Patent Application No. 10-2020-0110929, filed on Sep. 1, 2020, in the Korean Intellectual Property Office, the entire disclosure of which is incorporated herein by reference.

### BACKGROUND

### 1. Field

Example embodiments of the present disclosure relate to an apparatus and method for estimating a target component.

### 2. Description of Related Art

Diabetes is a chronic disease that causes various complications and is difficult to cure, and hence people with diabetes are advised to check their blood glucose regularly to prevent complications. In particular, when insulin is administered to control blood glucose, the blood glucose levels may need to be closely monitored to avoid hypoglycemia and control insulin dosage. There are several ways to monitor glucose levels, from invasive finger pricking testing to non-invasive glucose monitoring without causing pain. The invasive method may provide high reliability in measurement, but may cause pain and inconvenience as well as an increased risk of disease infections due to the use of injection. Recently, extensive research has been conducted on non-invasive measurements of blood glucose based on spectrum analysis without collecting blood.

### SUMMARY

One or more example embodiments provide an apparatus and method for estimating a target component.

Additional aspects will be set forth in part in the description which follows and, in part, will be apparent from the description, or may be learned by practice of the example embodiments of the disclosure.

According to an aspect of an example embodiment, there is provided an apparatus configured to estimate a target component, the apparatus including a spectrum acquisition device configured to acquire a training spectrum, and a processor configured to extract a principal component from the acquired training spectrum, determine an optimal number of principal components based on a size of the extracted principal component and a size of a residual, and generate a target component estimation model based on the determined optimal number of principal components.

The processor may be further configured to extract the principal component from the training spectrum through at least one of a principal content analysis (PCA), an independent component analysis (ICA), non-negative matrix factorization (NMF), and singular value decomposition (SVD).

The processor may be further configured to determine an order of the size of the residual based on comparing the size of the extracted principal component and the size of the residual, and determine the optimal number of principal components based on the determined order of the size of the residual.

The processor may be further configured to extract a principal component while increasing a number of principal components, compare the size of the extracted principal component and the size of the residual, and determine the optimal number of principal components based on the number of principal components at a point in time when the order of the size of the residual deviates from a lowest order.

The processor may be further configured to determine the optimal number of principal components by adding a predetermined value to the number of principal components at the point in time when the order of the size of the residual deviates from the lowest order.

The spectrum acquisition device may be further configured to acquire the training spectrum measured during a training interval in which a concentration of the target component is constant.

The training interval may include an interval in which a user is fasting.

The spectrum acquisition device may be further configured to receive the training spectrum from an external device.

The training spectrum may include at least one of a reflection spectrum, a single beam spectrum, a near-infrared absorption spectrum, and a mid-infrared absorption spectrum.

The near-infrared absorption spectrum may be in a range of 4000 to 5000 cm⁻¹.

The near-infrared absorption spectrum may be in a range of 5500 to 6500 cm⁻¹.

The processor may be further configured to generate, based on a net analyte signal (NAS), the target component estimation model by using a number of principal components corresponding to the determined optimal number of principal components.

The processor may be further configured to, based on the spectrum acquisition device acquiring an estimation spectrum, estimate the target component by using the generated target component estimation model based on the estimation spectrum.

The target component may include at least one of glucose, urea, lactate, triglyceride, total protein, cholesterol, collagen, elastin, keratin, and ethanol. According to another aspect of an example embodiment, there is provided a method of estimating a target component including acquiring a training spectrum, extracting a principal component from the acquired training spectrum, determining an optimal number of principal components based on a size of the extracted principal component and a size of a residual, and generating a target component estimation model based on the determined optimal number of principal components.

The extracting of the principal component may include extracting the principal component from the training spectrum through at least one of a principal content analysis (PCA), an independent component analysis (ICA), non-negative matrix factorization (NMF), and singular value decomposition (SVD).

The determining of the optimal number of principal components may include determining an order of the size of the residual based on comparing the size of the extracted principal component and the size of the residual, and determining the optimal number of principal components based on the determined order of the size of the residual.

The determining of the optimal number of principal components may include extracting a principal component while increasing a number of principal components, comparing the size of the extracted principal component and the size of the residual, and determining the optimal number of principal components based on the number of principal components at a point in time when the order of the size of the residual deviates from a lowest order.

The determining of the optimal number of principal components may include determining the optimal number of principal components by adding a predetermined value to the number of principal components at the point in time when the order of the size of the residual deviates from the lowest order.

The method may further include acquiring an estimation spectrum, and estimating the target component by using the generated target component estimation model based on the estimation spectrum.

### BRIEF DESCRIPTION OF THE DRAWINGS

FIG. 1 is a block diagram illustrating an apparatus for estimating a target component according to an exemplary embodiment;

FIGS. 2A, 2B, and 2C are diagrams for describing an example of determining the number of principal components;

FIG. 3 is a block diagram illustrating an apparatus for estimating a target component according to another exemplary embodiment;

FIG. 4 is a flowchart illustrating a method of estimating a target component according to an exemplary embodiment;

FIG. 5 is a flowchart illustrating a method of generating a target component estimation model according to an exemplary embodiment; and

FIG. 6 is a diagram illustrating a wearable device according to an exemplary embodiment.

### DETAILED DESCRIPTION

Details of exemplary embodiments are provided in the following detailed description with reference to the accompanying drawings. The disclosure may be understood more readily by reference to the following detailed description of exemplary embodiments and the accompanying drawings. The disclosure may, however, be embodied in many different forms and should not be construed as being limited to the exemplary embodiments set forth herein. Rather, these exemplary embodiments are provided so that the disclosure will be thorough and complete and will fully convey the present disclosure to those skilled in the art, and the disclosure will only be defined by the appended claims. Like reference numerals refer to like elements throughout the specification.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations such as “comprises” or “comprising,” will be understood to imply the inclusion of stated elements but not the exclusion of any other elements. Terms such as “unit” and “module” denote units that process at least one function or operation, and they may be implemented by using hardware, software, or a combination of hardware and software.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

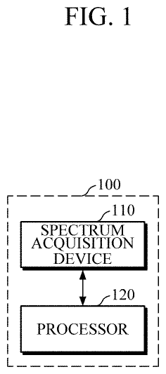

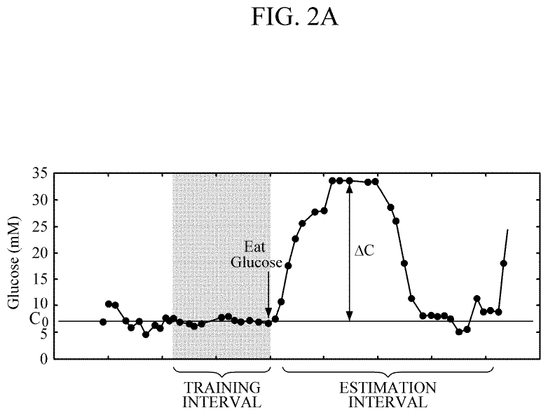

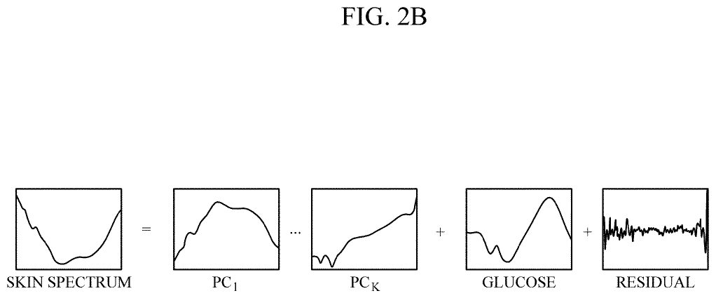

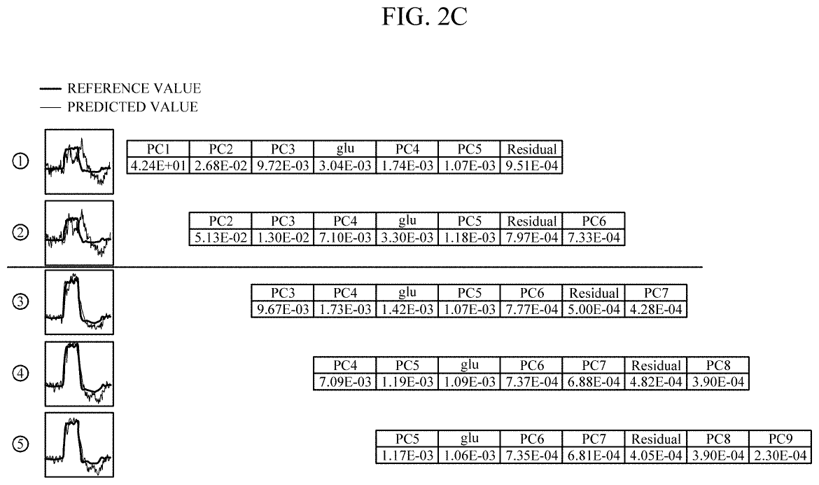

FIG. 1 is a block diagram illustrating an apparatus for estimating a target component according to an exemplary embodiment. FIGS. 2A to 2C are diagrams for describing an example of determining the number of principal components.

The apparatus for estimating a target component according to exemplary embodiments is an apparatus capable of analyzing a spectrum of an object and may be embedded in various electronic devices. Examples of the electronic devices may include mobile phones, smartphones, tablet computers, notebook computers, personal digital assistants (PDAs), portable multimedia players (PMPs), navigation systems, MP3 players, digital cameras, wearable devices, and the like, and the wearable devices may include wearable devices of a wristwatch type, a wrist band type, a ring type, a belt type, a necklace type, an ankle band type, a thigh band type, a forearm band type, and the like. However, the electronic devices are not limited to the aforementioned examples and the wearable devices are also not limited to the aforementioned examples.

Referring to FIG. 1, the apparatus 100 for estimating a target component includes a spectrum acquisition device 110 and a processor 120.

The spectrum acquisition device 110 may acquire a spectrum of an object. In this case, the spectrum of the object may include a reflection spectrum, a single beam spectrum, a near-infrared absorption spectrum, a mid-infrared absorption spectrum, and the like. For example, the near-infrared absorption spectrum may include a near-infrared absorption spectrum in the range of 4000 to 5000 cm⁻¹ or a near-infrared absorption spectrum in the range of 5500 to 6500 cm⁻¹. However, the near-infrared absorption spectrum is not limited thereto.

The spectrum acquisition device 110 may acquire an in vivo spectrum that is a training spectrum measured during a training interval in which the concentration of the target component of the object is substantially constant. In this case, the training interval may include a fasting interval. In addition, the spectrum acquisition device 110 may acquire an in vivo spectrum that is an estimation spectrum measured during an estimation interval in which the concentration of a target component of the object is changed, such as when a user eats food. In this case, the target component may include glucose, urea, lactate, triglyceride, total protein, cholesterol, collagen, elastin, keratin, ethanol, and the like. However, the target component is not limited to these examples, and may include various components formed by molecular bonds, for example, C—H, N—H, and O—H.

For example, the spectrum acquisition device 110 may acquire an in vivo spectrum from an external device that measures the in vivo spectrum, or from an external device that stores the in vivo spectrum. In this case, the external device may include various portable devices, such as smartphones, tablet computers, smart rings, smart earphones, smart watches, smart bands, and the like, or a server of a specialized medical institution.

The spectrum acquisition device 110 may use various communication technologies, such as Bluetooth communication, Bluetooth low energy (BLE) communication, near field communication (NFC), wireless local access network (WLAN) communication, ZigBee communication, infrared data association (IrDA) communication, Wi-Fi Direct (WFD) communication, ultra-wideband (UWB) communication, Ant+ communication, Wi-Fi communication, radio frequency identification (RFID) communication, 3G communication, 4G communication, and/or 5G communication.

In another example, the spectrum acquisition device 110 may emit light to the object and receive light reflected or scattered from the object to acquire a spectrum. In this case, the spectrum acquisition device 110 may use, but is not limited to, infrared spectroscopy or Raman spectroscopy, and may measure the spectrum using various spectral techniques.

The spectrum acquisition device 110 may include a light source configured to emit light to the object and a photodetector configured to receive light reflected or scattered from the object to measure the spectrum. Here, the light source may be configured to emit near infrared (NIR) light, mid-infrared (MIR) light, or the like to the object. However, the wavelength of the light emitted from the light source may vary depending on a purpose of measurement or the type of analyte. The light source may be configured as a single light-emitting body, or may be configured as a set of a plurality of light-emitting bodies. The light source may include a light emitting diode (LED), a laser diode, or a phosphor.

The photodetector may include a spectrometer configured to divide light reflected or scattered from the object to acquire a spectrum. Alternatively, the photodetector may be formed with a photo diode, a photo transistor, a complementary metal-oxide semiconductor (CMOS) image sensor, a charge-coupled device (CCD) image sensor, and the like. The photodetector may be configured as a single device, or may be configured as an array of a plurality of devices. The number and arrangement of the light sources and the photodetectors may vary according to the type of analyte, the application purpose, and the size and shape of the electronic device in which the apparatus 100 for estimating a target component is embedded.

The processor 120 may control the spectrum acquisition device 110 to generate a target component estimation model and to estimate a target component, and may receive a spectrum from the spectrum acquisition device 110. When a learning spectrum including one or more in vivo spectra is received from the spectrum acquisition device 110, the processor 120 may generate a target component estimation model using the learning spectrum. In addition, when estimation spectra, including one or more in vivo spectra, are received, the processor 120 may estimate the target component using the target component estimation model, on the basis of the estimation spectra.

For example, the processor 120 may generate a target component estimation model based on a net analyte signal (NAS) algorithm. For example, referring to FIG. 2A, based on the NAS algorithm, the target component estimation model may be generated by identifying a spectrum change factor that is relatively irrelevant to a change in a target component concentration by using, as training data, in vivo spectra measured during a training interval.

In addition, the processor 120 may estimate a target component, based on the NAS algorithm, using an in vivo spectrum measured from an estimation interval following the training interval and the target component estimation model. That is, in general, a target component estimation model is generated based on an in vivo spectrum measured during the training interval and then applied to the estimation interval to estimate a target component. Accordingly, when at least one of the spectrum change factors that are relatively irrelevant to a concentration change of the target component (e.g., blood glucose) is changed by any factor, such as a temperature change of an object, a change in pressure between the object and the device, or the like, at a specific point in time during the estimation interval, residual increases from the specific point in time, which may lead to increase of blood glucose estimation error.

The processor 120 may extract principle components that constitute the object from the training spectra, and generate the target component estimation model on the basis of the extracted principal components. For example, the processor 120 may extract the principle components through a principal component analysis. However, embodiments are not limited to the principal component analysis. For example, the principal component may be extracted using an independent component analysis (ICA), non-negative matrix factorization (NMF), singular value decomposition (SVD), and the like.

Referring to Equation 1 below and FIG. 2B, in the case of blood glucose estimation, a skin spectrum includes spectra of principal components (PC₁, . . . and, PCₖ), a blood glucose signal, and a residual spectrum.

PS=C₁·PC₁+ . . . +Cₖ·PCₖ+ε_{g}L·ΔC_{g}+RS [Equation 1]

Here, PS denotes an in vivo spectrum acquired from the object. PC₁ denotes the first principal component, and PCₖ denotes the k-th principal component. C₁ and Cₖ denote the size of the first principal component and the size of the k-th principal component, respectively. ε_{g}·L·ΔC_{g} denotes a blood glucose signal. ε_{g} denotes a unit spectrum for pure blood glucose, L denotes an optical path length, and ΔC_{g} denotes a variation in blood glucose compared to a reference blood glucose value. RS denotes a residual spectrum. ε_{g} and L may be measured experimentally in advance using water or a serum solution.

As described above, the skin spectrum may be expressed through the principal components. In general, if the number of principal components is too small, the skin spectrum may not be properly expressed, and if the number of principal components is too large, performance may be diminished in the estimation interval. Therefore, by generating the target component estimation model using the optimal number of principal components that are applicable not only to the training interval, but also to the estimation interval, the performance of estimating the target component may be improved.

To this end, the processor 120 may determine the optimal number of principal components that can be meaningfully applied in the training interval and the estimation interval by using the training spectrum acquired in the training interval, and may generate the target component estimation model using as many principal components as the determined optimal number of principal components.

For example, the processor 120 may extract a plurality of principal components from the training spectrum, and determine the optimal number of principal components by comparing the size of each of the extracted principal components and the size of residual. For example, referring to Equation 1, the size of each of the principal components may be defined as abs(Cₖ)*norm(PCₖ), wherein norm(PCₖ) is 1 and hence abs(Cₖ) is the size of the principal component. In addition, the size of residual may be defined as norm(RS) where, abs denotes the absolute value.

The processor 120 may extract the principal components while gradually increasing the number of principal components using the training spectra. FIG. 2C illustrates the arrangement of principal components in descending order of size, wherein the principal components are extracted while increasing the number of principal components from 5 to 9, and the principal components and the residual are arranged by comparing the sizes of the extracted principal components and the residual. As illustrated in FIG. 2C, the size of the first principal component may be the greatest and the sizes of subsequent principal components are determined according to the order of the principal components. In addition, as illustrated, the size of the residual, other than the target component signal, may lie between two principal components as the number of principal components is gradually increased.

The processor 120 may determine the optimal number of principal components on the basis of the order of the residual. For example, the optimal number of principal components may be determined on the basis of the number of principal components at a point in time when the order of the size of the residual deviates from the lowest order, that is, when one principal component having a size smaller than the size of the residual occurs.

For example, the processor 120 may determine the optimal number of principal components to be a value obtained by adding a predefined value, for example, 1, to the number of principal components at the time when the order of the size of the residual deviates from the lowest order. Here, the predefined value is not limited to 1, and the value may be defined according to characteristics of each user, computing performance of the apparatus 100, and the type of target component. Referring to \textcircled{2} in FIG. 2C, when the number of principal components is 6, the size of the residual lies before the size of the sixth principal component PC6. Accordingly, the optimal number of principal components may be determined to be 7 by adding 1 to the current number of principal components, i.e., 6. Referring to \textcircled{3} in FIG. 2C, when the number of principal components is 7, the performance of predicted blood glucose value is improved.

When the optimal number of principal components is determined, the processor 120 may generate a target component estimation model by using the relation of Equation 1 and a number of principal components corresponding to the optimal number of principal components. In addition, upon acquiring estimation spectra measured during the estimation interval, the processor 120 may estimate a target component by using the target component estimation model.

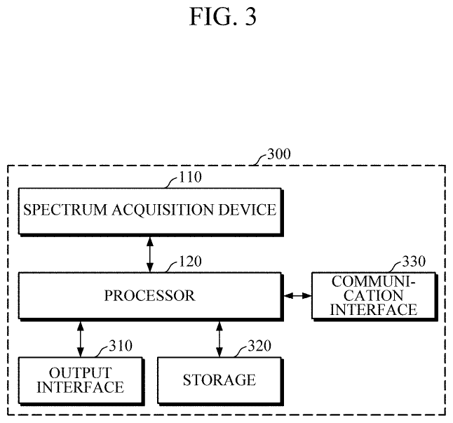

FIG. 3 is a block diagram illustrating an apparatus 300 for estimating a target component according to another exemplary embodiment.

Referring to FIG. 3, the apparatus 300 for estimating a target component may include a spectrum acquisition device 110, a processor 120, an output interface 310, a storage 320, and a communication interface 330. Here, the spectrum acquisition device 110 and the processor 120 have been described in detail with reference to FIG. 1, and thus detailed descriptions thereof will not be reiterated.

The output interface 310 may output data generated and processed by the spectrum acquisition device 110 and/or the processor 120. For example, the output interface 310 may output a spectrum acquired by the spectrum acquisition device 110 and/or an estimated target component value acquired by the processor 120, warning information according to whether or not the estimated target component value is normal, and the like. The output interface 310 may output an estimation result of a target component by using at least one of an acoustic method, a visual method, and a tactile method. The output interface 310 may include, for example, a display, a speaker, and a haptic module, such as a vibrator.

The storage 320 may store programs or commands for operation of the apparatus 300 for estimating a target component, and may store data input to the apparatus 300 and data related to target component estimation, for example, an in vivo spectrum, a target component estimation model, an estimated target component value, and the like.

The storage 320 may include at least one type of storage medium, such as a flash memory type, a hard disk type, a multimedia card micro type, a card type memory (e.g., a secure digital (SD) or eXtreme digital (XD) memory), a random access memory (RAM), a static random access memory (SRAM), a read-only memory (ROM), an electrically erasable programmable read-only memory (EEPROM), a programmable read-only memory (PROM), a magnetic memory, a magnetic disk, an optical disk, and the like. In addition, the apparatus 300 for estimating a target component may operate an external storage medium, such as a web storage that performs the storage function of the storage 320 via the Internet.

The communication interface 330 may communicate with an external device. For example, the communication interface 330 may transmit data input to, stored in, and processed by the apparatus 300 for estimating a target component to the external device, or may receive various types of data to generate and update a target component estimation model and to estimate a target component from the external device.

In this case, the external device may be, for example, a medical device that uses the data input to, stored in, and processed by the apparatus 300 for estimating a target component, or may be a printer or a display device to output a result. In addition, the external device may be a digital television (TV), a desktop computer, a mobile phone, a smartphone, a tablet computer, a notebook computer, a PDA, a PMP, a navigation system, an MP3 player, a digital camera, a wearable device, or the like, but is not limited thereto.

The communication interface 330 may communicate with the external device using a communication technology, such as Bluetooth communication, BLE communication, NFC, WLAN communication, ZigBee communication, IrDA communication, WFD communication, UWB communication, Ant+ communication, Wi-Fi communication, RFID communication, 3G communication, 4G communication, and 5G communication. However, these are examples, and the communication technology is not limited thereto.

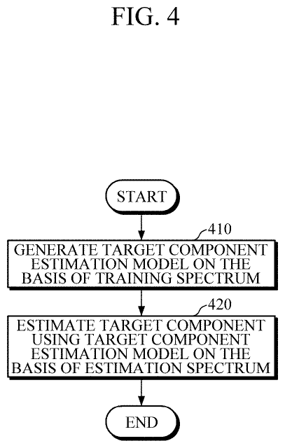

FIG. 4 is a flowchart illustrating a method of estimating a target component according to an exemplary embodiment.

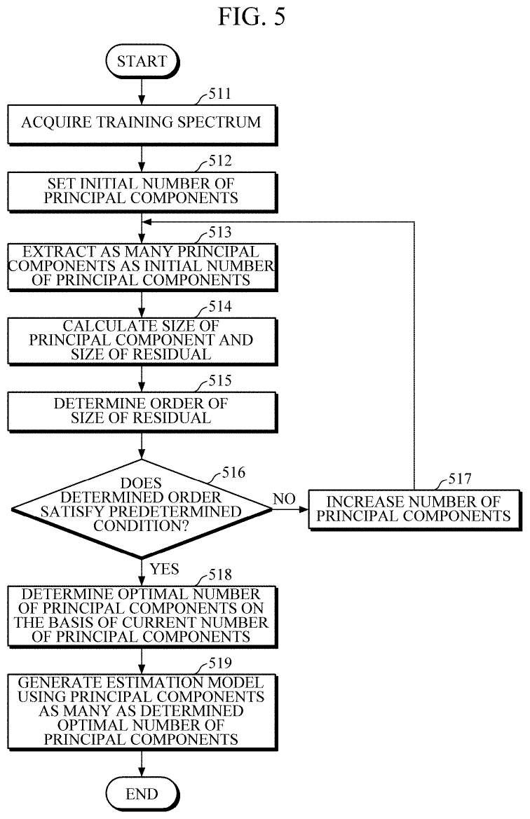

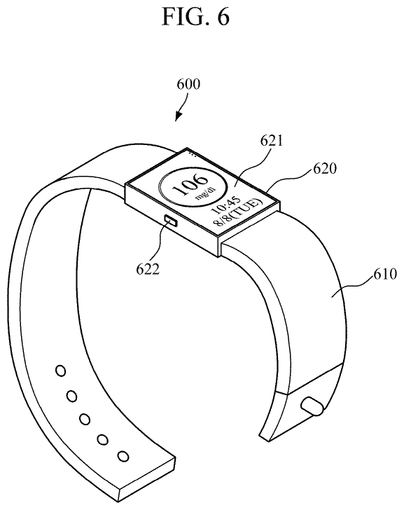

The method of FIG. 4 may correspond to a method performed by the apparatuses 100 and 300 of FIGS. 1 and 3, respectively, to estimate a target component. FIG. 5 is a flowchart illustrating a method of generating a target component estimation model according to an exemplary embodiment. FIG. 6 is a flowchart illustrating a method of estimating a target component according to another exemplary embodiment.

Referring to FIG. 4, in operation 410, a target component estimation model may be generated based on training spectra. The apparatus 100/300 for estimating a target component may acquire one or more training spectra and generate a target component estimation model based on a NAS algorithm. The apparatus 100/300 for estimating a target component may acquire the training spectra from an object of a user during a training interval, for example, a fasting interval, in which the target component is constant. The apparatus 100/300 for estimating a target component may extract principal components constituting the training spectra by using a PCA technique, and may determine the optimal number of principal components by comparing the size of the extracted principal components and the size of residuals. In addition, when the optimal number of principal components is determined, a target component estimation model may be generated, based on the NAS algorithm, by using the determined optical number of principal components.

An example embodiment of operation 410 of generating the target component estimation model will be described with reference to FIG. 5.

Referring to FIG. 5, in operation 511, the apparatus 100/300 for estimating a target component may acquire a training spectrum. As described above, in operation 511, the training spectra may be directly measured from the object of the user by using a sensor embedded in the apparatus for estimating a target component, or may be received from an external device. In this case, the training spectra may be acquired in a stable interval, in which the target component is constant, such as a fasting interval.

Then, in operation 512, the initial number of principal components may be set. In this case, the initial number of principal components may be predefined. For example, the initial number of principal components may be appropriately set based on the optimal number of principal components acquired at the time of a previous calibration.

Then, in operation 513, a number of principal components corresponding to the number of principal components set in operation 512 may be extracted from the training spectra acquired in operation 511. At this time, the principal components may be extracted through a PCA technique or the like.

Thereafter, in operation 514, the size of each of the extracted principal components and the size of a residual may be calculated, and in operation 515, the order of the size of the residual may be determined by comparing the calculated sizes of the principal components and the calculated size of the residuals.

Then, in operation 516, whether the order of the size of the residual satisfies a predetermined condition may be determined. For example, whether the order of the size of the residual deviates from the lowest order may be determined, and in operation 517, when the order of the size of the residual does not deviate from the lowest order, the number of principal components may be increased, and the procedure proceeds to operation 513 to extract a main component.

When it is determined in operation 516 that the order of the size of the residual satisfies the predetermined condition, in operation 518, the optimal number of principal components may be determined based on the current number of principal components. For example, the number obtained by adding a predetermined value (e.g., 1) to the current number of principal components may be determined to be the optimal number of principal components.

Then, in operation 519, a target component estimation model may be generated using as many principal components as the determined optimal number of principal components.

Referring back to FIG. 4, upon acquiring the estimation spectra from the object during the estimation interval, in operation 420, the apparatus 100/300 for estimating a target component may estimate the target component using the target component estimation model generated in operation 410. The estimated target component value may be provided to the user via the output interface, such as a display, a speaker, or the like.

FIG. 6 is a diagram illustrating a wearable device according to an exemplary embodiment. The above-described various example embodiments of the apparatuses 100 and 400 for estimating a target component may be embedded in, for example, a smart watch-type wearable device as illustrated. However, the embodiments are not limited thereto and may be embedded in a portable device, such as a smartphone.

Referring to FIG. 6, the wearable device 600 includes a device main body 620 and a strap 610.

The strap 610 may be connected to both ends of the main body 620 and enable the user to wear the main body 620 on his/her wrist. The strap 610 may be made of a flexible material to conform to the user's wrist. The strap 610 may consist of a first strap member and a second strap member that are separated from each other. Each of one ends of the first strap member and the second strap member may be connected to each of the both ends of the main body 620, and the first strap member and the second strap member may be fastened to each other via fastening means formed on the other ends thereof. In this case, the fastening means may be formed, for example, as a magnet fastening means, a Velcro fastening means, a pin fastening means, but is not limited thereto. In addition, the strap 610 may be formed as an integrated piece, such as a band.

The main body 620 may include various modules that perform various functions of the wearable device 600. A battery may be embedded in the main body 620 and the strap to supply power to various modules.

The main body 620 may be equipped with a spectrum acquisition device that acquires a spectrum from the user, and the spectrum acquisition device may include a light source and a photodetector as described above.

A processor may be mounted in the main body 620 and may be electrically connected to various components of the wearable device 600. The processor may determine the optimal number of principal components on the basis of training spectra acquired by the spectrum acquisition device, and may generate an estimation model for estimating a target component on the basis of NAS by using a number of principal components corresponding to the determined optimal number of principal components.

In addition, a storage may be mounted in the main body 620, and data related to a target component estimation function of the wearable device 600 and/or data related to other functions may be stored in the storage.

Also, a manipulator 622 may be mounted at one side of the main body 620 to receive a control command of the user and deliver it to the processor. The manipulator 622 may include a power button for inputting a command for turning on/off the wearable device 600.

In addition, a display 621 may be provided on the front surface of the main body 620 to output information, and the display 621 may include a touch screen capable of receiving touch input. The display may receive a user's touch input, transmit the received touch input to the processor, and display a processing result of the processor.

In addition, a communication interface that communicates with an external device may be mounted in the main body 620. The communication interface may transmit an estimation result of the target component to the external device, such as a smartphone of the user, and may receive data related to target component estimation from the external device.

The example embodiments may be implemented as computer readable codes in a computer readable record medium. Codes and code segments constituting the computer program may be easily inferred by a skilled computer programmer in the art. The computer readable record medium includes all types of record media in which computer readable data are stored. Examples of the computer readable record medium include a ROM, a RAM, a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner.

Various examples embodiments have been described above, but various modifications may be made. For example, suitable results may be achieved if the described techniques are performed in a different order and/or if components in a described system, architecture, device, or circuit are combined in a different manner and/or replaced or supplemented by other components or their equivalents.

While one or more example embodiments have been described with reference to the figures, it will be understood by those of ordinary skill in the art that various changes in form and details may be made therein without departing from the spirit and scope as defined by the following claims.

What is claimed is:

1. An apparatus configured to estimate a target component, the apparatus comprising:
a spectrum acquisition device configured to acquire a training spectrum; and
a processor configured to:
extract a principal component from the acquired training spectrum;
determine an optimal number of principal components based on a size of the extracted principal component and a size of a residual; and
generate a target component estimation model based on the determined optimal number of principal components.

2. The apparatus of claim 1, wherein the processor is further configured to extract the principal component from the training spectrum through at least one of a principal content analysis (PCA), an independent component analysis (ICA), non-negative matrix factorization (NMF), and singular value decomposition (SVD).

3. The apparatus of claim 1, wherein the processor is further configured to determine an order of the size of the residual based on comparing the size of the extracted principal component and the size of the residual, and determine the optimal number of principal components based on the determined order of the size of the residual.

4. The apparatus of claim 3, wherein the processor is further configured to:
extract a principal component while increasing a number of principal components;
compare the size of the extracted principal component and the size of the residual; and
determine the optimal number of principal components based on the number of principal components at a point in time when the order of the size of the residual deviates from a lowest order.

5. The apparatus of claim 4, wherein the processor is further configured to determine the optimal number of principal components by adding a predetermined value to the number of principal components at the point in time when the order of the size of the residual deviates from the lowest order.

6. The apparatus of claim 1, wherein the spectrum acquisition device is further configured to acquire the training spectrum measured during a training interval in which a concentration of the target component is constant.

7. The apparatus of claim 6, wherein the training interval includes an interval in which a user is fasting.

8. The apparatus of claim 1, wherein the spectrum acquisition device is further configured to receive the training spectrum from an external device.

9. The apparatus of claim 1, wherein the training spectrum includes at least one of a reflection spectrum, a single beam spectrum, a near-infrared absorption spectrum, and a mid-infrared absorption spectrum.

10. The apparatus of claim 9, wherein the near-infrared absorption spectrum is in a range of 4000 to 5000 cm⁻¹.

11. The apparatus of claim 9, wherein the near-infrared absorption spectrum is in a range of 5500 to 6500 cm⁻¹.

12. The apparatus of claim 1, wherein the processor is further configured to generate, based on a net analyte signal (NAS), the target component estimation model by using a number of principal components corresponding to the determined optimal number of principal components.

13. The apparatus of claim 1, wherein the processor is further configured to, based on the spectrum acquisition device acquiring an estimation spectrum, estimate the target component by using the generated target component estimation model based on the estimation spectrum.

14. The apparatus of claim 1, wherein the target component includes at least one of glucose, urea, lactate, triglyceride, total protein, cholesterol, collagen, elastin, keratin, and ethanol.

15. A method of estimating a target component comprising:
acquiring a training spectrum;
extracting a principal component from the acquired training spectrum;
determining an optimal number of principal components based on a size of the extracted principal component and a size of a residual; and
generating a target component estimation model based on the determined optimal number of principal components.

16. The method of claim 15, wherein the extracting of the principal component comprises extracting the principal component from the training spectrum through at least one of a principal content analysis (PCA), an independent component analysis (ICA), non-negative matrix factorization (NMF), and singular value decomposition (SVD).

17. The method of claim 15, wherein the determining of the optimal number of principal components comprises determining an order of the size of the residual based on comparing the size of the extracted principal component and the size of the residual, and determining the optimal number of principal components based on the determined order of the size of the residual.

18. The method of claim 17, wherein the determining of the optimal number of principal components comprises:
extracting a principal component while increasing a number of principal components;
comparing the size of the extracted principal component and the size of the residual; and
determining the optimal number of principal components based on the number of principal components at a point in time when the order of the size of the residual deviates from a lowest order.

19. The method of claim 18, wherein the determining of the optimal number of principal components comprises determining the optimal number of principal components by adding a predetermined value to the number of principal components at the point in time when the order of the size of the residual deviates from the lowest order.

20. The method of claim 15, further comprising:
acquiring an estimation spectrum; and
estimating the target component by using the generated target component estimation model based on the estimation spectrum.
